(19) United States
(12) Patent Application Publication
(10) Pub. No.: US 2024/0310649 A1
(43) Pub. Date: Sep. 19, 2024

(54) SYSTEM FOR TREATING PERSONALITY AND MOOD DISORDERS

(71) Applicant: Alejandro CONTADOR BLUMENBERG, Santiago (CL)

(72) Inventor: Alejandro CONTADOR BLUMENBERG, Santiago (CL)

(21) Appl. No.: 18/120,815

(22) Filed: Mar. 13, 2023

(51) Int. Cl. G02C 5/00; G02C 7/16

(52) U.S. Cl. CPC G02C 5/001; G02C 7/16; A61M 2021/0027; A61M 2021/0044

(57) ABSTRACT

A system for treating personality and mood disorders, comprising: at least one frame, comprising: at least two arms connected at the right and left sides of the at least one glass frame; at least two lens spaces disposed at the front right and left sides of the at least one frame, wherein at least one of the at least two lens spaces is divided into at least two visual spaces right and left; at least one blocking element disposed on at least one visual space of the at least one of the at least two lens spaces, which decreases the visibility of said at least one visual space; and at least one audio device, which reproduces at least one sound piece in at least one ear, corresponding to the opposite side in which is placed the at least one blocking element.

### FIELD OF THE INVENTION

The present invention relates to a system that combines different devices that include modified glasses and a headset that emits audio in a single ear to treat mental health disorders like personality and mood disorders.

Specifically, personality disorders are an enduring and inflexible pattern of long duration leading to significant distress or impairment and is not due to the use of substances or other medical conditions.

Personality disorders like avoidant personality disorder, Borderline personality disorder, Narcissistic personality disorder, Obsessive-compulsive personality disorder and other personality disorders can be effectively treated with this system.

It also treats a variety of mood disorders like depression and bipolar disorders.

More particularly the invention relates to a system that comprises two different types of glasses, that permit only see half right or left of visual field and a headset that emits audio in the right or left ear.

One device allows you to only see the right side of the visual field and the other device only allows you to see the left side of the visual field. To be able to look straight ahead, the person must turn their neck to the right or to the left.

The audio headset is placed over the ear on the side where the vision is forced to see. In other words, it is used over the ear on the opposite side from which the vision is blocked.

This is done according to which side should be stimulated according to each user.

Also, music or a conversation must be incorporated in one ear. If you want to stimulate the left hemisphere of the brain, you should put the headset over your right ear. If you want to stimulate the right hemisphere, you should put the headset over the left ear. The listening part is vital for the treatment because through language we interpret a large part of reality and thus we understand how to relate to other people. For this reason, the patient must first be tested to determine which cerebral hemisphere is predominant.

Once it is certain which cerebral hemisphere predominates, treatment can begin with either the type A or B device and a headset that emits audio in a single ear, used simultaneously.

### BACKGROUND

The increase in mental pathologies of all kinds and the limited effectiveness of psychological treatments is a well-known fact.

On the other hand, Pharmacological treatments are widely used, but they try to moderate the immediate and specific effects of the ailment and do not get to the root of the mental illnesses.

Psychotherapy, or talk therapy, may help in managing personality disorders. During psychotherapy, you and a therapist can discuss your condition, as well as your feelings and thoughts. This can provide you with insight on how to manage your symptoms and behaviors that interfere with your daily life.

This is why psychological therapies of all kinds, which have a great variety, are still used today and although they have limited effects on mood disorders and low efficacy on personality disorders.

Today there is practically a consensus that personality disorders do not have a solution and talk therapies only allow patients to become aware of the distortions of reality and thus take actions that allow them to better adapt to the environment.

Personality disorders are not treatable, however. Often a combination of talk therapy and medication can go a long way in helping patients live with one of these conditions.

On the other hand, the system of the present invention blocks the entry of visual and auditory information from one cerebral hemisphere and sends all the audio and vision through the opposite hemisphere, being specifically to treat mental illness like personality and mood disorders.

This is done through two types of devices modified glasses that block and blind half of the visual field, either on the left or right side, in this way, it diverts the vision to the right or left side. To be able to look straight ahead, the person must turn their neck either to the right or to the left, to stimulate the left or right hemisphere, depending on the side of the brain that is decompensated.

It is necessary to place an audio headset on the side where the vision is being directed or guided. In other words, it is used over the ear on the opposite side from where the vision is blocked.

The listening part is vital for the treatment because through language we interpret a large part of reality and thus we understand how to relate to other people.

According to the literature, for treating personality disorders, it must change how the person thinks and to do that it is necessary to change how the person perceives reality. People capture an important part of reality in the listening part, by hearing other people speak. Therefore, the person being treated must receive the audio in a single cerebral hemisphere, depending on which hemisphere needs to be stimulated.

This is done according to which side should be stimulated according to each user.

These differentiation abilities and functions of the hemispheres are well known, and to summarize, it is known that the left hemisphere is dominant in speech and logic and the right hemisphere is more skilled in spatiality and emotionality.

When the difference of one hemisphere over another is very marked, the point of view of the other hemisphere is lost or diminished, creating character structure problems, such as the psychopath who only sees his interest and point of view or, on the contrary, the dependent personality disorder who overestimates others to the detriment of their interest and opinion.

Hemisphere imbalance has much deeper consequences than just abilities, they determine the character structure since, ultimately, these are human and mammal evolutionary survival strategies in general.

That is why we find dogs with what we could call different characters, some very aggressive and others extremely fearful, even being siblings.

Cerebral hemispheres imbalance causes mental illness.

In this regard, human beings, like the other mammals that have two cerebral hemispheres; allows them to have perspective of the facts or reality, perspective is another point of view.

In the same way, we need two eyes to know how far away objects are and two ears to know where a sound is coming from.

If one of the cerebral hemispheres has an excessive predominance in interpretation, reality is tinged with that trend, creating rigid and repetitive behavior patterns that over the years cause mood disorders, that are so common these days.

The brain's general function is to capture reality and with the glasses and headset of the present invention, the information of reality is processed in a single hemisphere.

The principle is like exercise, but for the cerebral.

With this treatment, the user begins to see results after a few weeks, preferably after three months, but to improve or heal a personality disorder and/or a profound behavioral modification can be noted after one year of treatment.

In this regard, there are some solutions in the field of patents which intend to solve this problematic. One of them is related to the U.S. Pat. No. 5,424,786, whose objective is to improve reading comprehension. The main differences with the present invention are:

a.—As the document literally says:
The masked portion reduces visuals stimuli entering the side of the person's eyes that to the hemisphere of the brain in which the visual stimuli is to be reduced.
Clearly in the images two types of hatchings or latticework are shown, one with a striped design and the other with dots. both designed to reduce visual stimuli.

The present invention has several devices that include two different types of glasses that completely block or blind half of your eyes visual field, in this way, it directs or guides your vision completely either to the right or to the left side and an audio headset.

In fact, to be able to look straight ahead, the person must turn their neck either to the right or to the left.

b.—In the patent document, it is mentioned only glasses. In the present invention, it is a combination of glasses and audio that allow a complete, integral and deep auditory and visual stimulation of the brain.

c.—In the patent document, the vision controlling device causes the left hemisphere to be available to assist the right hemisphere with the task, “therefore increasing the reading capability of such a person”. The goal is to improve reading comprehension.

The present invention has a different objective, such as the treatment of personality disorders, which is a psychiatric condition.

d.—The patent document literally says: It is advantageous for a right hemisphere dominant person to reduce the natural visual stimuli going to the left hemisphere. If the left side is dominant, the stimulus in the right hemisphere is reduced.

The present invention is the opposite of that, if the right hemisphere is dominant, the visual stimulation to the same right hemisphere is blocked. If the left is dominant, the left hemisphere is blocked. It does not only reduce it is totally blocked. In other words, the present patent application stimulates the opposite cerebral hemisphere than the document of the state of the art says.

Another similar patent corresponds to U.S. Pat. No. 5,963,294, which describes a method for using therapeutic glasses for stimulating a change in the psychological state of a subject.

The main differences with the present invention are:

a. The document aims to change the psychological state of the person, but in the present patent application, the specific objective is the treatment of personality disorders.

b. The document restricts the vision of only one eye, leaving the other eye unchanged, In the present patent application, half of the visual field of both eyes are completely blocked, directing or guiding the vision either to the right or left and to look straight ahead you must rotate your neck.

c. The present patent application includes a listening device on the opposite side of where the glasses are blocked, this audio is necessary for the audio/visual stimulation for the right or left hemisphere, and thus, it modifies the verbal thinking and auditory comprehension of reality necessary to treat personality disorders.

d.—The patent document literally says; the subject can switch between embodiments to alter the lateral visual field which is stimulated. The person uses both types of glasses interchangeably. In the present patent application, cerebral dominance must be previously determined and the stimulus is blocked only for the dominant cerebral hemisphere. Each patient uses only one type of glasses, right or left, never both.

### BRIEF SUMMARY OF THE INVENTION

The present invention is directed to the combination of devices that treats a wide range of mental illnesses. This is done by means of two types of modified glasses that the vision is being directed or guided to look only at the visual field to the right or left side and a headset that emits audio in a single ear, in this respect, the left or right hemisphere of the brain is stimulated as needed.

Specifically, the present invention teaches a system for treating personality and mood disorders, comprising:
at least one frame, comprising:
at least two arms connected at the right and left sides of the at least one glass frame;
at least two lens spaces disposed at the front right and left sides of the at least one frame, wherein at least one of the at least two lens spaces is divided into at least two visual spaces right and left;
at least one blocking element disposed on at least one visual space of the at least one of the at least two lens spaces, which decreases the visibility of said at least one visual space; and
at least one audio device, which reproduces at least one sound piece in at least one ear, corresponding to the opposite side in which is placed the at least one blocking element.

According to another embodiment of the invention, the at least one blocking element is disposed on the right visual spaces of the at least two lens spaces.

According to another embodiment of the invention, the at least one audio device reproduces the at least one sound piece in the left ear.

According to another embodiment of the invention, the at least one blocking element is disposed on the left visual spaces of the at least two lens spaces.

According to another embodiment of the invention, the at least one audio device reproduces the at least one sound piece in the right ear.

According to another embodiment of the invention, the system further comprises at least two lens disposed on the at least two lens spaces.

According to another embodiment of the invention, the at least one blocking element is disposed over the at least two lens in the at least one visual space of the at least one of the at least two lens spaces.

According to another embodiment of the invention, the at least one blocking element blocks the visibility on the at least one visual space in the range comprised between 10 to 95%, preferably between 50 to 60%.

In other words, the system allows the visibility of the user in the range comprised between 5 to 90%, preferably between 40 to 50%.

According to another embodiment of the invention, the at least one blocking element is made of cardboard, laminating sheet, plastic, glass, metal, or from a combination of them.

According to another embodiment of the invention, the at least one blocking element comprises at least one smooth sheet without holes.

According to another embodiment of the invention, the at least one blocking element comprises at least one vertical slot.

According to another embodiment of the invention, the at least one blocking element comprises at least one horizontal slot.

According to another embodiment of the invention, when the system is used by a user, he can do an exercise of raising and lowering his view. Specifically, the user can do the exercise of raising and lowering his view at intervals of around 6 seconds, preferably of around 4 seconds, and more preferably of around 2 seconds. The purpose of this action is directed to speed up the healing process of the user.

Finally, according to another embodiment of the invention, it is described the use of a system for treating personality and mood disorders, wherein it is it is used by a user, which carry on an exercise of raising and lowering his view at intervals of around 6 seconds, preferably of around 4 seconds, and more preferably of around 2 seconds.

### BRIEF DESCRIPTION OF THE DRAWINGS

FIG. 1. It is a perspective view of two types of glasses, type A forces the vision to the right side of visual field and type B device forces the vision to the left side of visual field.

FIG. 2. It is a front view of two types of modified glasses, type A and B, in a real sample (wearing) of how they look from the front. Device type A forces the vision to the right (from the wearer's point of view) and device type B forces the vision to the left.

FIG. 3. It is a top view of two types of glasses, seen from above and show their respective fields of vision. Device type A forces the vision to the right (from the wearer's point of view) and device B forces the vision to the left.

FIG. 4. It is a view from behind of two types of glasses, that completely block the view of one half of the eye and thus force the vision to the opposite side.

FIG. 5. It is a top view of two types of glasses, type A and B. Here it shows how the image of the type A device is captured and processed on the right hemisphere of the brain and the same for the type B device.

FIG. 6. It is an aerial view showing an audio-emitting headset worn in either the left or right ear.

FIG. 7. In this front view you can see how the system is used with glasses and headset at the same time. In its two versions depending on the side that needs to be blocked and stimulated on the contrary.

### DETAILED DESCRIPTION OF THE INVENTION

This invention relates to a system which comprises modified glasses and a headset that emits audio used simultaneously to treat non-organic mental illnesses, personality and mood disorders.

Human beings interpret reality mainly through sight and hearing, with taste, touch and smell having less relevance in mental effort.

However, the interpretation of the reality of sight and hearing is really made by the brain, having two different interpretations; one made by the left hemisphere and the other by the right.

In this sense, human beings, like other mammals that have two cerebral hemispheres; allows them to have perspective of the facts or reality, perspective is another point of view.

In the same way, we need two eyes to know how far away objects are and two ears to know where a sound is coming from.

If one of the cerebral hemispheres has an excessive predominance in interpretation, reality is tinged with that trend, creating rigid and repetitive behavior patterns that over the years cause mood disorders, that are so common these days.

The solution is to completely block the sight and audio of one of the cerebral hemispheres and force all the visual and auditory information to enter the weakened hemisphere, in order to train it as any muscle or organ is trained to improve its influence on the interpretation of reality.

In this respect, the mind's character is centered and modified.

This is done through a system which can comprise two types of glasses that divert the vision to the right or left side, together with a headset that transmits audio that can be either music or conversation to stimulate the left or right hemisphere, depending on the side of the brain that is decompensated.

For the treatment of personality and mood disorders, the person must wear the modified glasses and the headset at the same time.

The general function of the brain is to capture reality and with this combination of devices, one hemisphere is blocked, and all information of reality is processed in the other hemisphere.

In this regard, the brain works in a similar way to a muscle, therefore, to improve the hemispheric imbalance of the brain, it must be trained, but in this case only the atrophied hemisphere must be trained.

That said, it must be determined and established for sure which is the side of the atrophied brain, in order to focus on its training.

Two types of Mechanical therapy glasses are used for this.

Modified eyeglass devices in which the visual field of the left or right side of each eye is covered.

The first pair of glasses stimulate the left hemisphere, Type A in all Fig. allows to only see 40% to the right side and the left side of each eye is blocked, 60% covered and does not allow visibility in the right or left eye.

The second pair of glasses stimulate the right hemispheres of the brain, type B in all Fig., this device allows visibility in both eyes (left and right), but only to the left side. enable or allow 40% vision.

Said in simpler words, it forces the vision to the left side and to be able to look straight ahead, the person must turn their neck either to the right or to the left.

The right side of each eye is covered 60% and does not allow visibility in either the right or left eye.

It is necessary to place an audio headset on the side where the vision is being directed or guided. In other words, it is used over the ear on the opposite side from where the vision is blocked.

The listening part is vital for the treatment because through language we interpret a large part of reality and thus we understand how to relate to other people.

“To treat personality and mood disorders, we must change how the person thinks and to do that we need to change how the person perceives reality. We capture an important part of reality in the listening part, by hearing people speak. Therefore, the person being treated must receive the audio in a single cerebral hemisphere, depending on which hemisphere needs to be stimulated”.

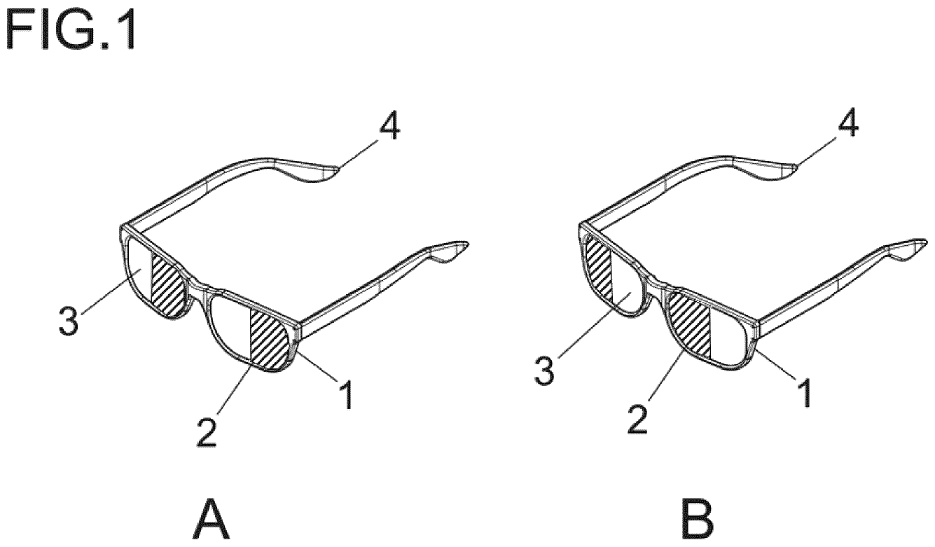

FIG. 1.

FIG. 1 shows a perspective view of the devices, which are two different lenses but use the same operating principle, that is, they cover or block half the visual field of the eyes, in this way, they force the vision of both eyes either to the right or to the left of the visual field.

Type A glasses only allow visibility to the right visual field of both eyes. the two left halves, (Num. 2). They are completely covered and block visibility, covering it. (Num. 3). indicates that you are allowed to see freely through that part of the lens, but forcing your vision to the right.

The eyeglass lenses can be glass, mica, or nothing at all, that is irrelevant for it to work, it only matters that they block half the field of vision, so they force visibility to the right.

The frame of the glasses indicated in (Num. 1) can be made of any material or shape, since it does not influence its functionality.

The arms of the glasses, which are indicated in (Num. 4), can be of any size, material or shape and do not affect their functionality.

Type B glasses only allow visibility to the right visual field of both eyes. the two left halves, (Num. 2) of FIG. they are completely covered and block visibility, covering it. (Num. 3) in Fig. indicates that you are allowed to see freely through that part of the lens, but forcing your vision to the left.

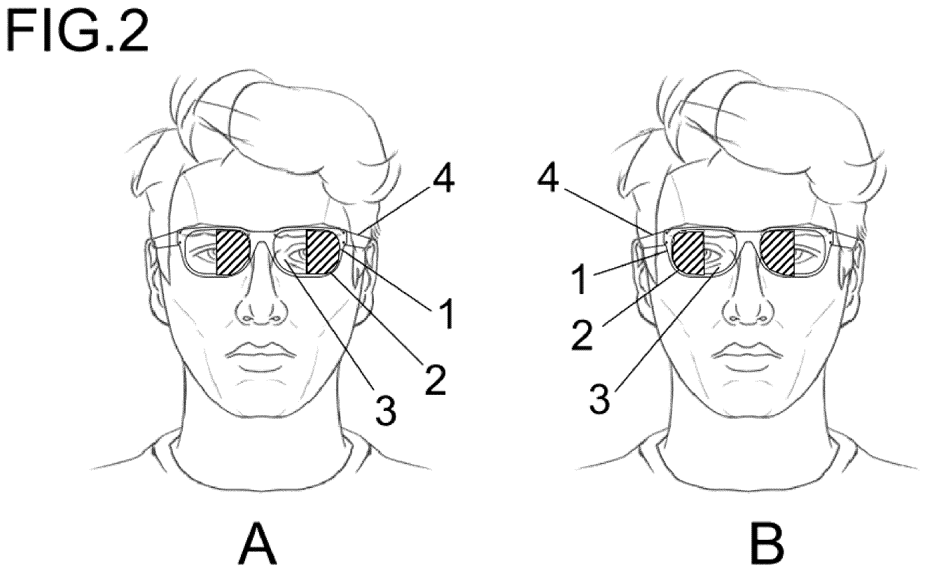

FIG. 2.

It represents a frontal view of the devices (Num. 4) in use, on a person's ace (Num. 1).

They are two types of glasses, but they use the same principle, completely blocking the view of half the visual field and thus forcing visibility only to the opposite side. ‘The type A glasses forces the view to the right side of the visual field and the type B forces the view to the left side of the visual field.

The glasses block 60% of the visual field. (Num. 2) and force the vision to the opposite side but allow 40% visibility of the visual field. (Num. 1)

Type A blocks the left side of the visual field and stimulates the left hemisphere of the brain. Type B blocks the right visual field and stimulates the right hemisphere of the brain.

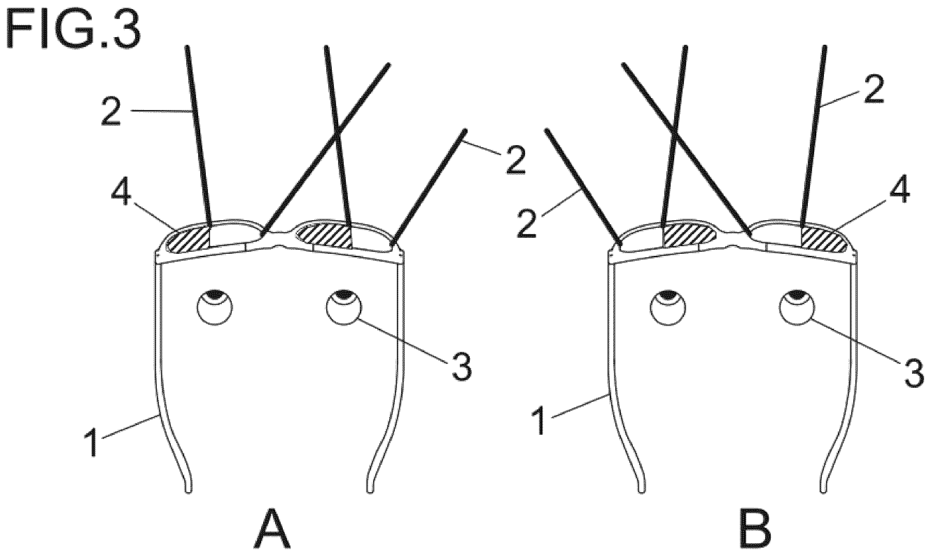

FIG. 3.

This is an image seen from above. The objective is to demonstrate the visual field blocked (Num. 4) and the visual field that can be seen (Num. 2). In (Num. 3) the eyes are shown and (Num. 1) shows the modified glasses

The type A device blocks the view of the right half of the visual field (Num. 4) and forces visibility only to the right side of the visual field, (Num. 2).

The type B device blocks the view of the left half of the visual field (Num. 4) and forces visibility only to the left side of the visual field, (Num. 2).

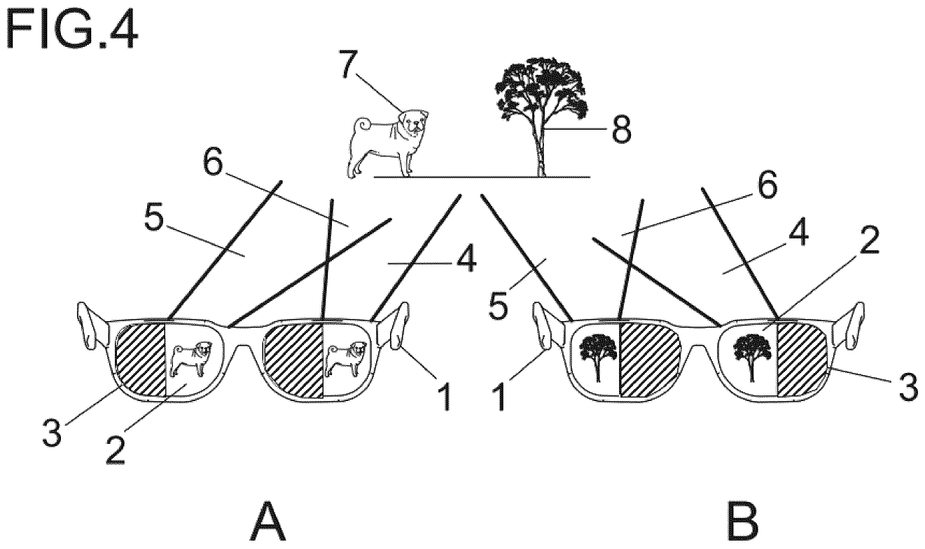

FIG. 4.

In this image, we see the glasses from behind.

As in the previous images, we see type A glasses that block the visual field on the left and force or obligate us to look to the right.

In type A glasses, with forced vision to the right, it allows us to only see only the image of the dog, since this image is in the permitted visual field.

The left side of the device is blocked, with 60% of no visibility in the left visual field.

In (Num. 7) we see an image of a dog, in (Num. 5) the visual field of the left eye, in (number 4) the visual field of the right eye is shown, in (Num. 6) the intersection of the visual field of both eyes. The (Num. 1) shows the glasses, (Num. 3) the blocked part of the visibility and finally the (Num. 2) shows the image of the dog that is seen only in the right half of the visual field of both eyes.

And B glasses block the gaze of the visual field on the right, forcing the view to the left of the visual field.

In FIG. It is observed that it allows to see only the image of the tree, which is in the permitted visual field.

In (Num. 8) we see the image of the tree, in (Num. 5) the visual field of the left eye, in (Num. 4) the visual field of the right eye is shown, in (Num. 6) the intersection of the field sight of both eyes.

The (Num. 1) shows the glasses, the (Num. 3) the blocked part of the visibility and finally the (Num. 2) shows the image of the tree that is seen only in the left half of the visual field of both eyes.

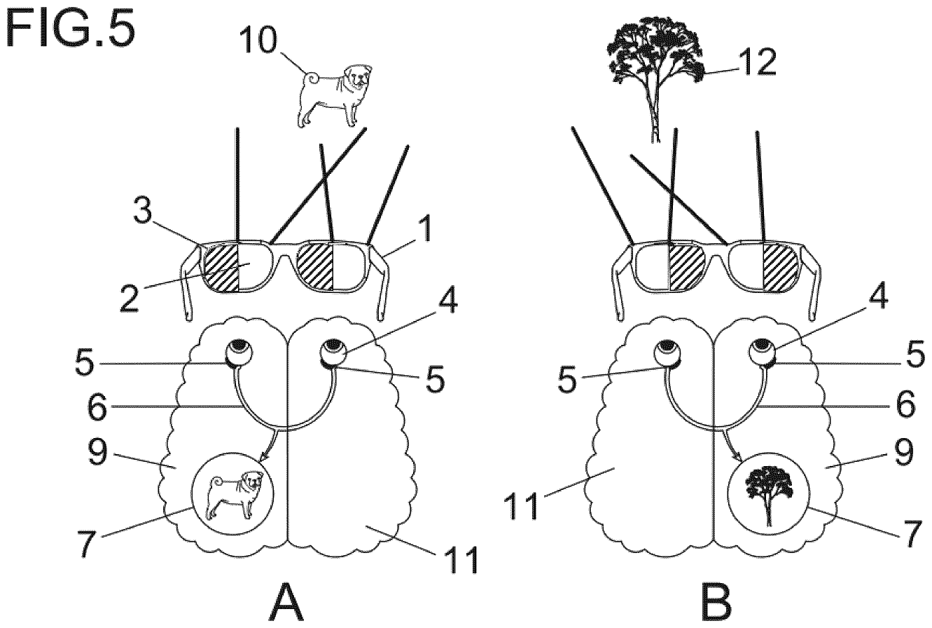

FIG. 5.

This figure shows the path taken by an image captured by the eye until it is processed in one hemisphere of the brain, specifically in the occipital lobe, left or right, depending on the glasses used.

This occurs because when using glasses, the vision is forced to the left or right, the vision is forced to the right or to the left, and thus the images are processed in the right or left cerebral hemisphere. While keeping the vision to one side over time, the opposite hemisphere to which one is looking develops.

The type A glasses is shown with (Num. 1). The (Num. 3) shows the membrane that blocks the vision of the left visual field in both eyes. The (Num. 2) shows the free field of vision, but in order to look you must force your gaze to the right. the (Num. 10) shows the image of a dog that can only be seen by forcing the gaze to the right side of the visual field.

The (Num. 11) shows the left side of the brain, which is stimulated by the information processing in that hemisphere and the (Num. 9) shows the right hemisphere of the brain, which is blocked since no visual information reaches it. (Num. 4) shows the eyes and (Num. 5) shows the left side of the retina, this is very important because by forcing the gaze to the right all the light and information arrive only on the left side of the retina, and through the optic nerve indicated in (Num. 6) reaches the left hemisphere and the image is processed in the left occipital lobe indicated in (Num. 7).

It can also be seen in (Num. 7) how the image of the dog shown in (number 10) reaches the left posterior hemisphere.

If a person looks normally without these glasses, all images are processed in both hemispheres of the brain simultaneously.

Type B which are modified glasses is shown in (Num. 1). The (Num. 3) shows the cover that blocks the vision of the right side of the eyes. The (Num. 2) shows the free field of vision but that forces or forces to look to the left. The (Num. 12) shows the image of a star that can only be seen by straining your vision to the left side of the visual field.

The (Num. 11) shows the left side of the brain and the (number 9) shows the right side of the brain. (Num. 4) shows the eyes and (Num. 5) shows the right side of the retina, this is important because by forcing the vision to the left all the light and information reach only the right side of the retina, and this side right passes the information captured by the eye through the optic nerve indicated in (Num. 6).

The information reaches the right hemisphere and is processed mainly in the occipital lobe indicated in (Num. 7). It can be seen in (Num. 7) how the image of the star shown in (Num. 12) reaches the left rear hemisphere.

When people see normally, information is processed in both hemispheres of the brain. because no one in the world wears glasses to see only towards the left side of the visual field.

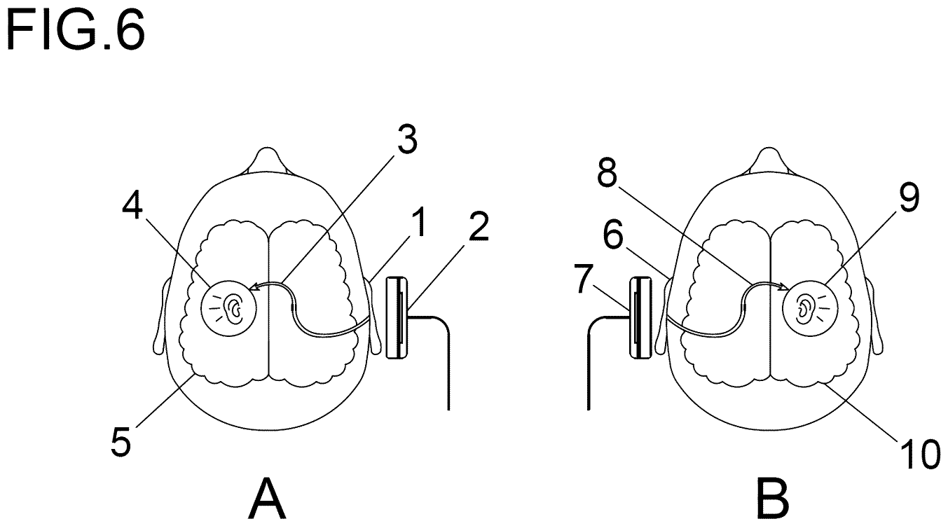

FIG. 6.

It is a top view showing a dissection of the brain. Type A is seen in which a headset emits music or conversation of the patient's taste is used in the right ear and this audio is processed in the left hemisphere, specifically in the auditory cortex, which is part of the temporal lobe and processes the information. Hearing in humans and other vertebrates.

(Num. 1) shows the ear, (Num. 2) indicates the headset that emits audio, which can be either music or conversation, (Num. 3) shows the cochlear nerve, (Num. 4) indicates the position of the auditory cortex, in charge of interpreting the meaning of sound and (Num. 5) shows the left hemisphere.

In type B we see how the headset is placed in the left ear and the audio is interpreted in the right hemisphere of the brain, also in the auditory cortex, part of the temporal lobe.

(Num. 6) shows the ear, (Num. 7) indicates the earphone that emits audio, which can be either music or conversation, (Num. 8) shows the cochlear nerve, (Num. 9) indicates the position of the auditory cortex, in charge of interpreting the meaning of sound and the (Num. 10) shows the left hemisphere.

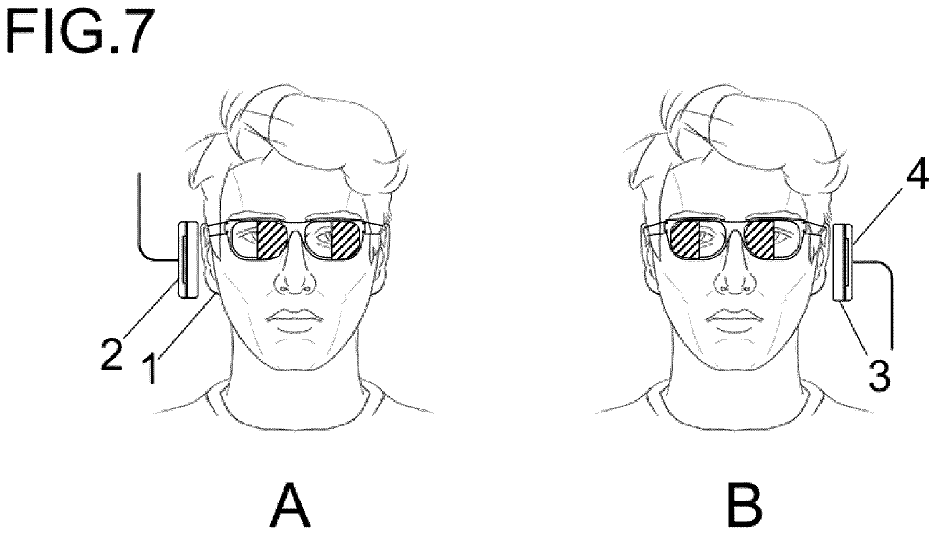

FIG. 7.

It is a frontal image that indicates the system summary in which all the visual and auditory information in one hemisphere is blocked and the entire flow of visual and auditory information is saturated or sent to the opposite cerebral hemisphere.

In type A, the right hemisphere is blocked and all the information flow is sent to the left hemisphere. (Num. 1) shows the ear and (Num. 2) the headset that transmits audio, which can be anything that fulfills the function.

In type B, the left hemisphere is blocked and all the information flow is sent to the right hemisphere. (Num. 3) shows the ear and (Num. 4) the headset that transmits audio, which can be anything that fulfills the function. The content can be any music or conversation that the patient likes to make it more pleasant for them.

1. A system for treating personality and mood disorders, comprising:
at least one frame, comprising:
at least two arms connected at the right and left sides of the at least one glass frame;
at least two lens spaces disposed at the front right and left sides of the at least one frame, wherein at least one of the at least two lens spaces is divided into at least two visual spaces right and left;
at least one blocking element disposed on at least one visual space of the at least one of the at least two lens spaces, which decreases the visibility of said at least one visual space; and
at least one audio device, which reproduces at least one sound piece in at least one ear, corresponding to the opposite side in which is placed the at least one blocking element.

2. The system for treating personality and mood disorders of claim 1, wherein the at least one blocking element is disposed on the right visual spaces of the at least two lens spaces.

3. The system for treating personality and mood disorders of claim 2, wherein the at least one audio device reproduces the at least one sound piece in the left ear.

4. The system for treating personality and mood disorders of claim 1, wherein the at least one blocking element is disposed on the left visual spaces of the at least two lens spaces.

5. The system for treating personality and mood disorders of claim 4, wherein the at least one audio device reproduces the at least one sound piece in the right ear.

6. The system for treating personality and mood disorders of claim 1, wherein it further comprises at least two lens disposed on the at least two lens spaces.

7. The system for treating personality and mood disorders of claim 6, wherein the at least one blocking element is disposed over the at least two lens in the at least one visual space of the at least one of the at least two lens spaces.

8. The system for treating personality and mood disorders of claim 1, wherein the at least one blocking element blocks the visibility on the at least one visual space in the range comprised between 10 to 95%, preferably between 50 to 60%.

9. The system for treating personality and mood disorders of claim 1, wherein the at least one blocking element is made of cardboard, laminating sheet, plastic, glass, metal, or from a combination of them.

10. The system for treating personality and mood disorders of claim 1, wherein the at least one blocking element is formed from at least one smooth sheet without holes.

11. The system for treating personality and mood disorders of claim 1, wherein the at least one blocking element comprises at least one vertical slot.

12. The system for treating personality and mood disorders of claim 1, wherein the at least one blocking element comprises at least one horizontal slot.

13. The system for treating personality and mood disorders of claim 1, wherein when it is used by a user, he can do an exercise of raising and lowering his view.

14. The system for treating personality and mood disorders of claim 13, wherein the exercise of raising and lowering the view of the user can be done at intervals of around 6 seconds, preferably of around 4 seconds, and more preferably of around 2 seconds.

15. Use of a system for treating personality and mood disorders according with claim 1, wherein it is it is used by a user, which carry on an exercise of raising and lowering his view at intervals of around 6 seconds, preferably of around 4 seconds, and more preferably of around 2 seconds.
